(19) United States
(12) Patent Application Publication
(10) Pub. No.: US 2020/0224166 A1
(43) Pub. Date: Jul. 16, 2020

(54) METHOD FOR PREPARING HETEROGENEOUS HEMATOPOIETIC STEM AND PROGENITOR CELLS USING NON-MOBILIZED PERIPHERAL BLOOD

(71) Applicant: ZHEJIANG UNIVERSITY, Hangzhou City (CN)

(72) Inventors: He HUANG, Hangzhou City (CN); Yulin XU, Hangzhou City (CN); Xiaohong YU, Hangzhou City (CN)

(21) Appl. No.: 16/740,489

(22) Filed: Jan. 13, 2020

Related U.S. Application Data
(63) Continuation of application No. PCT/CN2019/094977, filed on Jul. 8, 2019.

(30) Foreign Application Priority Data
Jan. 16, 2019 (CN) 201910039866.6

(51) Int. Cl. C12N 5/0789; C12Q 1/6806; C12N 15/10; C12Q 1/6876; G01N 33/50; A61K 49/00

(52) U.S. Cl. CPC C12N 5/0647; C12N 2501/999; C12N 15/1096; C12Q 1/6876; G01N 33/5005; A61K 49/0008; C12N 2533/30; C12N 2501/125; C12N 2501/998; C12N 2501/145; C12N 2501/2306; C12N 2501/2303; C12N 2501/165; C12N 2500/38; C12Q 1/6806

(57) ABSTRACT

The present disclosure provides a method for preparing heterogeneous hematopoietic stem and progenitor cells using non-mobilized peripheral blood, which uses a capsule culture system to capture and proliferate rare hematopoietic stem and progenitor cells in non-mobilized peripheral blood, and prepares heterogeneous hematopoietic stem and progenitor cell clones. The present disclosure captures the rare heterogeneous stem cells in non-mobilized peripheral blood and morphologically verifies the presence of heterogeneous hematopoietic stem and progenitor cells in non-mobilized peripheral blood. The method of the present disclosure has the characteristics of hematopoietic reconstitution, drug development, transplantation and immunotherapy, gene editing of cell types, and the like. The method of the present disclosure provides a reliable cell source for patient-specific functional hematopoietic stem cells, and actively promotes the clinical application of non-mobilized hematopoietic stem and progenitor cells.

### TECHNICAL FIELD

The present disclosure belongs to biological technologies, relates to biological technologies such as cell biology, non-mobilized peripheral blood, heterogeneous hematopoietic stem and progenitor cells, and cell capturing, culturing, proliferation, function maintaining and the like, and in particular, relates to a method for preparing heterogeneous hematopoietic stem and progenitor cells using non-mobilized peripheral blood.

More specifically, the present disclosure provides a technical system for capturing and proliferating rare heterogeneous hematopoietic stem and progenitor cells in non-mobilized peripheral blood and also provides cell source having the characteristics of hematopoietic reconstitution, drug development, transplantation and immunotherapy, and gene editing.

### BACKGROUND

Cancer has become the number one killer of human health. In 2015, there were more than 21 million new cancer cases every year in the world, and China had 4.292 million new cases and 2.814 million cancer deaths, accounting for about 20% of the global new cases and equivalent to an average of 12,000 new cancer cases and 7,500 cancer deaths every day. In the United States, 1,685,210 new cancer cases were diagnosed in 2016, of which 595690 died of this type of diseases. The latest cancer data from China in March 2017 shows that approximately 10,000 people are diagnosed with cancer every day in China; approximately 7 people are diagnosed with cancer every minute; by the age of 85, one person has a 36% risk of cancer, in addition, it is expected that the number of cancer patients in China is increased year by year to 19 million people by 2025, and cancer cases in Asia increase by about 40% by 2030 and reach 24 million people by 2035 with a mortality rate increase of about 50%. Cancer prevention and treatment have become important public health issues in China and the world.

Radio-chemotherapy and surgery are currently the main methods for treating cancer. Surgical treatment mainly targets solid tumors without metastases. For tumor patients from whom tumors cannot be removed completely by surgery and patients in middle and late stages, radio-chemotherapy is one of the most effective treatment methods to save and prolong the lives of patients. Whether it is chemotherapy, surgery or radiotherapy, the treatment of cancer is a huge burden on the body, and it is difficult to completely cure the cancer in any way after the malignant metastasis occurs. So the treatment of cancer is still a great test for human beings.

After high-dose radio-chemotherapy, hematologic systems of patients, such as the immune cells, are severely damaged, and hematopoietic stem cell transplantation becomes one of the important means of tumor treatment supporting high-dose radio-chemotherapy. At present, its application range is increasingly wide, including a variety of malignant tumors and hematological malignant tumors, etc. The malignant tumors include breast cancer, ovarian cancer, testicular cancer, neuroblastoma, and small cell lung cancer, etc. Malignant hematological diseases include chronic granulocytic leukemia, acute myeloid leukemia, acute lymphoblastic leukemia, non-Hodgkin's lymphoma, Hodgkin's lymphoma, multiple myeloma, and myelodysplastic syndrome, etc. Non-malignant hematological tumors are mainly myelofibrosis, aplastic anemia, megakaryocyte-free thrombocytopenia, thalassemia, Fanconi anemia, sickle cell anemia, and severe paroxysmal nocturnal hemoglobinuria, etc. Other non-hematological diseases are mainly severe refractory autoimmune diseases, such as severe combined immunodeficiency, severe autoimmune diseases and the like. Hematopoietic stem cell transplantation has gradually become an important means for treatment of various diseases including tumors.

Hematopoietic stem cell transplantation researches began in 1939, but the first transplantation test was unsuccessful. After nearly forty years of extensive discussion, animal experiments and re-evaluation, human beings have gradually gained a deeper understanding of bone marrow transplantation. The first large-scale allogeneic hematopoietic stem cell transplantation began in 1975. From then on, the hematopoietic stem cell transplantation began to play an important role in the human anticancer history. At present, cells for hematopoietic stem and progenitor cell transplantation are mainly derived from mobilized peripheral blood hematopoietic stem and progenitor cells (derived from bone marrow) and umbilical cord blood hematopoietic stem and progenitor cells. Umbilical cord blood-derived hematopoietic stem and progenitor cells are small in number and have delay in reconstitution of a hematopoietic system, and do not meet the requirements of transplantation number of adult clinical hematopoietic stem and progenitor cells. The current clinically used cells are mainly derived from mobilized peripheral blood, containing hematopoietic stem and progenitor cells derived from bone marrow. In this case, the donor needs to continuously take a mobilizing drug, granulocyte colony-stimulating factor G-CSF, granulocyte macrophage colony-stimulating factor GM-CSF, or the like, for about a week to mobilize the hematopoietic stem and progenitor cells in the bone marrow into peripheral blood, and then, the hematopoietic stem and progenitor cells are collected by using a cell collector for cell transplantation. However, the hematopoietic stem and progenitor cells used for transplantation need to be successfully matched with patients, and the patients need to take immunosuppressants for a long time to reduce the graft-versus-host disease (GVHD) response. In view of the low success rate of hematopoietic stem cell matching, the severe shortage of hematopoietic stem cell source has restricted the widespread and effective application of this technology in clinical practice; and long-term taking of immunosuppressants has also brought patients with risks such as relapses, infections, and secondary tumors, etc. In addition, blood sources such as red blood cells and platelets that are needed clinically are highly tight in supply, including storage and use of rare blood types and the like. Blood source pollution and blood product borne diseases are all global challenges. Exploring new hematopoietic stem and progenitor cells and the source of patient-specific hematopoietic stem and progenitor cells is an urgent problem.

In the 1950s, researchers introduced lymphoid leukocytes in peripheral blood into radiated animals, the granulocyte systems of the radiated animals recovered to a certain degree after a period of time, and the lives of the radiated animals were protected and prolonged to a certain extent. In 1957, Congdons C. C. and other researchers found that when the lymphoid leukocytes in peripheral blood were transplanted to animals radiated at a lethal dose, the survival rate of the animals was closely correlated with the number of transplanted cells. In 1968, Lewis and other researchers transplanted the lymphoid leukocytes in peripheral blood into mice, nodules could be found in the spleens of the transplanted mice after a period of time, and further experiments showed that cells in these nodules came from the transplanted lymphoid peripheral blood, proving that this group of lymphoid cells have a multi-lineages differentiation potential of bone marrow-derived hematopoietic stem and progenitor cells.

The above research history is mainly based on transplantation of lymphoid cells in the peripheral blood of animals of the same kind in animal bodies, and the number of spleen nodules formed in the transplanted animals was detected so as to detect the possibility of the presence of hematopoietic stem and progenitor cells in peripheral blood. There were relatively more studies in the 1960s and 1970s. However, on the one hand, due to the extremely small number of hematopoietic stem and progenitor cells in peripheral blood (called circulating hematopoietic stem and progenitor cells), there has been no effective capture, maintenance, and proliferation system so far, therefore, whether this group of cells are present in normal human peripheral blood or not is still a big dispute, and the biological characteristics of circulating hematopoietic stem and progenitor cells are even more blank. There are few related research reports now. At present, discussions mainly focus on the role and function of circulating endothelial cells on progression stages in a disease state. The main technical means is only detection by flow cytometry and clone forming experiments, but there is no effective capture, proliferation and culture system to further explore the biological characteristics of circulating hematopoietic stem and progenitor cells, especially, to estimate self-renewal and differentiation abilities.

Because the hematopoietic stem and progenitor cells play an important role in tumor treatment, hematopoietic immune reconstitution, gene therapy, aging delay, etc., exploring the capture, proliferation, and culture of functional circulating hematopoietic stem and progenitor cells and the function maintenance thereof will bring huge economic and social benefits.

### SUMMARY

An object of the present disclosure is to provide a method for preparing heterogeneous hematopoietic stem and progenitor cells using non-mobilized peripheral blood. Rare hematopoietic stem and progenitor cells in non-mobilized peripheral blood are captured using a capsule culture system. On this basis, heterogeneous hematopoietic stem and progenitor cell clones are prepared, hematopoietic stem and progenitor cells are proliferated, and the biological functions of the hematopoietic stem and progenitor cells are maintained. The above method is realized by the following technical schemes:

### 1. Source and Preparation of Initiating Cells

The initially cultured cells are derived from normal peripheral blood without mobilizing drug treatment. The amount of blood is not limited, and may be less than 1 ml, or more than 1 ml. The blood can be collected at a specific amount as needed. Erythrocytes are removed from the obtained blood product by using a lymphocyte separation solution or an erythrocyte lysing solution, and the obtained mononuclear cells are washed 2 to 3 times with a calcium ion and magnesium ion-free phosphate buffer solution, and used as the cells to be initially cultured for preparation, culture and capture of hematopoietic stem and progenitor cells.

### 2. Capture and Preparation of Heterogeneous Hematopoietic Stem and Progenitor Cell Clones

The above obtained mononuclear cells are capsuled with a cell culture material with an appropriate degree of softness and hardness, including but not limited to a hydrogel, and are seeded, which is called a capsule culture system. The cells is washed once with a 10% sucrose solution, re-suspended with 20% sucrose, mixed with the material according to a certain cell density, and seeded in a corresponding well plate. The cells are cultured in a culture system suitable for growth of hematopoietic stem and progenitor cells, containing a stem cell growth factor SCF (20-150 ng/ml), an FMS-like tyrosine kinase 3 ligand antibody (20-150 ng/ml), thrombopoietin TPO (20-100 ng/ml), interleukin 6 IL6 (10-50 ng/ml), interleukin 3 IL3 (10-50 ng/ml), a vascular growth factor VEGF (2-10 ng/ml), vitamin C (Vc, 10-20 ug/ml), and a puromycin derivative StemRegenin1 (SR1), etc. The culture medium is replaced every 2 to 3 days. After about 5 days of culture, most of the differentiated terminal blood cells in blood gradually die, and different clones in morphology begin to appear in the capsule cell culture system. With the culture time going on, the clones gradually proliferate. These clones include dense clones, vascular-like clones, cobble-stone-like clones, freely dispersed cells, etc. By contrast, in the non-capsule cell culture under the same conditions, namely, in a system that target cells are not capsuled with a material such as hydrogel and other culture conditions are the same, the various cell clones described above are not generated.

### 3. Detection of Heterogeneity of Hematopoietic Stem and Progenitor Cell Clones by Single Cell Sequencing

According to morphological characteristics, single cells in different clones are selected for single cell sequencing. Single-cell RNAs are extracted, mRNAs are enriched with magnetic beads with Oligo (dT), and cDNAs are synthesized using fragmented mRNAs as templates. A kit is used for purification and recovery, and a library is constructed by PCR amplification; the constructed library is used for sequencing, and the transcription expression of single-cell sequencing is detected. According to the number of reads obtained by gene sequencing, gene expression, optimization of genetic structure, alternative splicing, prediction and annotation of new transcripts, SNP detection, etc. are analyzed, genes that are differentially expressed among samples are screened out from the gene expression analysis, and GO function significance enrichment analysis and pathway significance enrichment analysis are performed based on the differentially expressed genes. Cell cluster analysis of principal components of single cells is performed to detect the heterogeneity of the above various clones.

### 4. Surface Molecule Expression of Heterogeneous Hematopoietic Stem and Progenitor Cell Clones

The various clones in the capsule culture system grow to a certain size, and each clone contains about 30 to 80 cells. The system is dispersed, mixed, and digested with an ethylenediamine tetraacetic acid digestive solution, passes through a 70 um mesh sieve, and is centrifuged to harvest cells. The surface molecule expression of hematopoietic stem and progenitor cells in the obtained cells is detected using flow cytometry, including CD34, CD43, CD45, and CD90, etc.

### 5. Detection of In Vitro Differentiation Potential

The clones of several cell types appearing in the capsule culture system, including dense clones, vascular-like clones, cobble-stone-like clones, freely dispersed cells, etc., are selected according to the cellular morphologies, 200 to 300 targeted cells are sorted and selected and CFU formation experiment is conducted in a growth factor-containing methylcellulose semi-solid medium, and the multi-lineages differentiation potential of different clones is detected, including burst erythroid colonies, generally small erythroid colonies, granulocyte colonies, granulocyte-macrophage colonies, erythroid-granulocyte-macrophage mixed cell colonies, etc. CD34⁺ cells sorted from normal mobilized peripheral blood served as positive control groups.

### 6. Detection of Growth Potential of Cells in Capsule Culture System

Non-mobilized peripheral blood cells are equally cultured in capsule and non-capsule culture systems. Growth potential analysis is conducted in a medium containing hematopoietic stem and progenitor cell growth factor, and the self-renewal potential of the targeted cells is estimated.

### 7. Detection of Expression of Transcription Factors of Hematopoietic Stem Cells in Capsule Culture System

Biological characteristics of the whole cell population in the capsule cell culture system are studied at a molecular level. Change of the cells in the capsule culture system at a transcriptome level is detected through RNA sequencing, especially hematopoietic stem cell-related transcription factors, signaling pathways and microenvironment-related regulating genes. The transcription factors include CD34, RUNX1, GATA2, c-MYC, HOXA9, HOXB4, GATA1, TIE2, etc., the signaling pathways mainly include genes regulating self-renewal, multi-lineages potential and metabolism state. The microenvironment-related regulating genes are mainly homing, cell adhesion related genes, etc.

### 8. Analysis of In Vivo Hematopoietic Differentiation Potential of Whole Cell Population in Whole Capsule Cell Culture System

The cell population with various hematopoietic colonies is dispersed and is prepared for the transplantation experiment. With intramedullary injection. Long-term self-renewal and multi-lineage differentiation potential in vivo are analyzed. Chimerism is detected at indicated time. Cells that are cultured in a non-capsuled manner with the same conditions are used as a control group.

The method provided by the present disclosure is characterized by using the capsule culture system to capture and proliferate the rare hematopoietic stem and progenitor cells in non-mobilized peripheral blood, and by producing heterogeneous hematopoietic stem and progenitor cell clones. The system captures the rare functionally heterogeneous stem cells in non-mobilized peripheral blood for the first time, and the presence of heterogeneous hematopoietic stem and progenitor cells are verified according to different clones in various morphologies in the non-mobilized peripheral blood for the first time. The method provides a reliable cell source for patient-specific functional hematopoietic stem cells, and actively promotes the clinical application of non-mobilized hematopoietic stem and progenitor cells.

### BRIEF DESCRIPTION OF DRAWINGS

FIG. 1 shows a technical process for obtaining hematopoietic stem and progenitor cells using non-mobilized peripheral blood. Non-mobilized peripheral blood of a donor is drawn. Mononuclear cells are obtained, the mononuclear cells are treated and cultured in a capsule culture system, and the presence and growth of clones of different morphologies are detected periodically.

FIG. 2 shows that clones of different morphologies presenting in non-mobilized peripheral blood, and the morphologies change obviously.

FIG. 3 shows that kinetic change for the surface marker expression of hematopoietic stem and progenitor cells in the capsule culture of non-mobilized peripheral blood cells with flow cytometry analysis.

FIG. 4 shows comparison of the clone formation ability between cells generated in capsule culture of non-mobilized peripheral blood and hematopoietic stem cells sorted from mobilized peripheral blood.

FIG. 5 shows comparative analysis of growth of cells after capsule culture and non-capsule culture of non-mobilized peripheral blood.

FIG. 6 shows a schematic diagram to analyze long-term in vivo self-renewal and multi-lineage differentiation potential of cells after capsule culture and non-capsule culture of non-mobilized peripheral blood.

FIG. 7 shows detection of T cells produced from the transplanted cells of the capsule culture and non-capsule culture of non-mobilized peripheral blood.

FIG. 8 shows detection of myeloid cells produced from the transplanted cells of the capsule culture and non-capsule culture of non-mobilized peripheral blood.

FIG. 9 shows detection of B cells produced from the transplanted cells of the capsule culture and non-capsule culture of non-mobilized peripheral blood.

FIG. 10 shows detection of human Th1 cells produced from the transplanted cells of the capsule culture and non-capsule culture of non-mobilized peripheral blood.

FIG. 11 shows detection of human Th2 cells produced from the transplanted cells of the capsule culture and non-capsule culture of non-mobilized peripheral blood.

FIG. 12 shows a flow chart of detection of human cells in peripheral blood of the transplanted mice injected with the cells of capsule culture of non-mobilized peripheral blood.

FIG. 13 shows a flow chart of detection of human cells in bone marrow of the transplanted mice injected with the cells of capsule culture of non-mobilized peripheral blood.

FIG. 14 shows a flow chart of detection of human cells in liver of the transplanted mice injected with the cells of capsule culture of non-mobilized peripheral blood.

FIG. 15 shows a flow chart of detection of human cells in spleen of the transplanted mice injected with the cells of capsule culture of non-mobilized peripheral blood.

FIG. 16 shows detection of long-term self-renewal and differentiation abilities of the cells in capsule culture of non-mobilized peripheral blood, and detection of human cells in peripheral blood for the second transplantation.

FIG. 17 shows detection of long-term self-renewal and differentiation abilities of the cells in capsule culture of non-mobilized peripheral blood, and detection of human cells in bone marrow for the second transplantation.

FIG. 18 shows detection of long-term self-renewal and differentiation abilities of the cells in capsule culture of non-mobilized peripheral blood, and detection of human cells in liver for the second transplantation.

FIG. 19 shows detection of long-term self-renewal and differentiation abilities of the cells in capsule culture of non-mobilized peripheral blood, and detection of human cells in spleen for the second transplantation.

FIG. 20 shows detection of expression of key hematopoietic transcription factors in non-mobilized peripheral blood after capsule culture and non-capsule culture, non-mobilized peripheral blood and mobilized hematopoietic stem cells.

FIG. 21 shows comparative analysis of expression of key transcription factors, signal pathways, etc. in capsule cultured non-mobilized peripheral blood, non-capsule cultured non-mobilized peripheral blood, non-mobilized peripheral blood, and mobilized hematopoietic stem cells using a single cell fluorescent quantitative PCR technology.

FIG. 22 shows that the ultrastructures of intracellular organelles are observed using a transmission electron microscopy. The mobilized hematopoietic stem cells serve as a positive control. Nucleo-cytoplasmic ratio in capsule culture and in non-capsule culture increase. A large number of endoplasmic reticulum, active mitochondria, and crest folds are observed during the capsule culture and the non-capsule culture.

### DESCRIPTION OF EMBODIMENTS

The present disclosure is further described with reference to the accompanying drawings and examples. The materials, reagents, etc. used in the following examples are commercially available unless otherwise specified.

### Example 1 Preparation of Heterogeneous Hematopoietic Stem and Progenitor Cell Clones by Using Non-Mobilized Peripheral Blood

It was briefly described as follows:

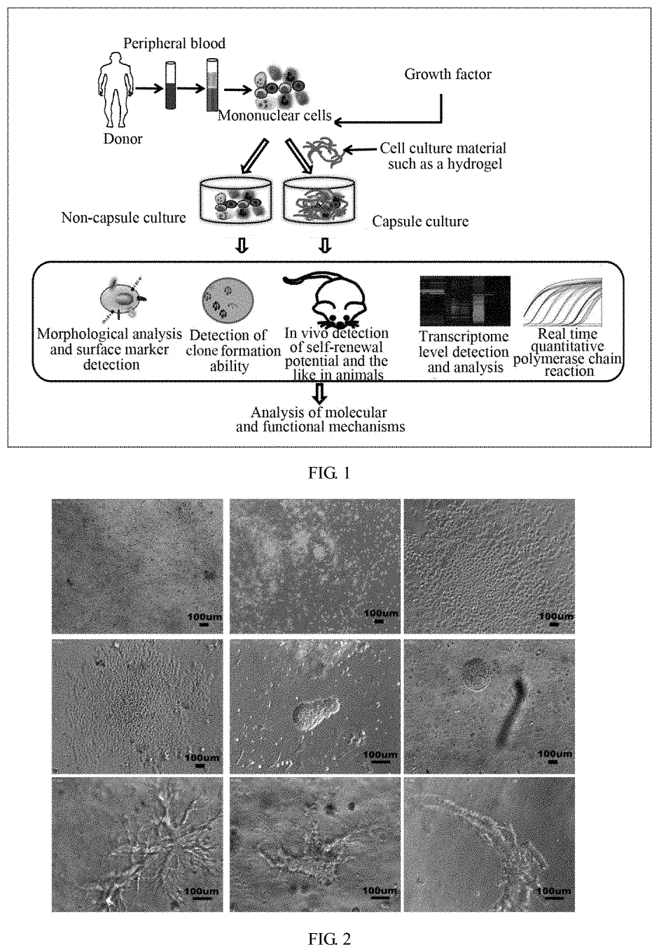

1. The present invention provides a method for capturing rare stem cells in non-mobilized peripheral blood by using a capsule culture system, and preparing heterogeneous hematopoietic stem and progenitor cell clones. On the basis of obtaining the heterogeneous hematopoietic stem and progenitor cell clones, a large number of hematopoietic stem and progenitor cells can be obtained by using a small amount of non-mobilized peripheral blood, and can be continuously used for downstream molecule and cell biological function analysis. The specific scheme is shown in FIG. 1.

### 2. Obtaining of Mononuclear Cells by Using Non-Mobilized Peripheral Blood

Volunteers were recruited. According to experimental needs, less than 1 ml or more than 1 ml of a blood product could be drawn aseptically and collected by using aseptic anticoagulation tubes.

2.1 Lysing of Erythrocytes with Erythrocyte Lysing Solution

Erythrocytes were lysed by using an erythrocyte lysing solution, 2 to 4 ml of the lysing solution was added per 1 ml of non-mobilized peripheral blood to lyse for 5 to 8 min on ice, and the change in color of the blood product was observed; when the blood product changed from original deep red to pale red in color, and gradually changed from original opacity to transparency, a suitable amount of calcium ion and magnesium ion-free phosphate buffer solution was added for neutralization, mononuclear cells were obtained by centrifuging at 1500 rpm for 5 min, and washed 2-3 times with the calcium ion and magnesium ion-free phosphate buffer solution, and the obtained mononuclear cells were subjected to the next experiment.

2.2 Separation of Mononuclear Cells from Blood Product by Using Lymphocyte Separation Solution

A lymphocyte separation solution and non-mobilized peripheral blood were added to a centrifuge tube according to a ratio of 1:2 to be centrifuged at 2500 rpm for 25 min at 4° C., a middle buffy coat was aspirated and washed 2-3 times with the calcium ion and magnesium ion-free phosphate buffer solution, and the obtained mononuclear cells were subjected to the next experiment.

### 3. Preparation of Heterogeneous Hematopoietic Stem and Progenitor Cell Clones

The mononuclear cells obtained from the non-mobilized peripheral blood were capsuled with a hydrogel with a moderate degree of softness and hardness, and the cells were enveloped in the material to be shaped like a capsule, which was called a capsule culture system. A serum-free hematopoietic stem cell proliferation medium SFEM (STEMCELL TECHNOLOGY) containing 20-200 ng/ml SCF, 20-200 ng/ml FLT3L, 10-20 ng/ml IL-3, 10-20 ng/ml IL-6, 10-100 ng/ml TPO, 2-10 ng/ml VEGF, and 5-30 ng/ml vitamin C was used for culturing, and the medium was replaced every 2 days. The growth statuses of cells and clones were observed under a microscope. The growth morphologies of clones and changes thereof were recorded. The morphological changes are shown in FIG. 2.

### 4. Detection of Expression of Hematopoietic Stem and Progenitor Cell-Related Surface Markers in Capsule Culture System by Flow Cytometry

After the clones grew to a certain size, the cloned cells were dispersed, washed with a buffer solution, and the expression of hematopoietic stem and progenitor cell expressing markers in the capsule culture system was detected by flow cytometry.

The details were as follows:

After the clones in the capsule culture system grew to about 50-80 um, the whole system was gently pipetted with a pipette tip to decompose the capsule system, centrifuging was performed to collect cells, the cells were digested with 0.25% trypsin/ethylenediamine tetraacetic acid for 10 min, the digestion was terminated by using a bovine serum-containing medium, and the cells were gently pipetted, passed through a 70 um cell filter, and centrifuged at 1000 rpm for 5 min to collect the cells. The cells were washed 2-3 times with the calcium ion and magnesium ion-free phosphate buffer solution to be collected. The cell density was adjusted to 10⁶-10⁷ cells per milliliter, the cells were added with corresponding flow antibodies, including CD34, CD45, CD43, CD90, CD309, CD117, CD19, CD15, CD3, etc., incubated at room temperature in a dark place for 30 min, and washed 2-3 times with the phosphate buffer solution, the cells were re-suspended with 500 uL of a phosphate buffer solution (added with 1% FBS and 1 mM ethylenediamine tetraacetic acid), and expression of multiple hematopoietic cell surface antigens in the capsule culture system was detected by a BD FACScalibur instrument (Becton Dickinson). The isotype Ig was used as a control. Data was analyzed by a software FlowJo Version 7.2.5. The statistical analysis results of flow detection are shown in FIG. 3.

### 5. Detection of In Vitro CFU Forming Potential of the Cells in Capsule Culture System

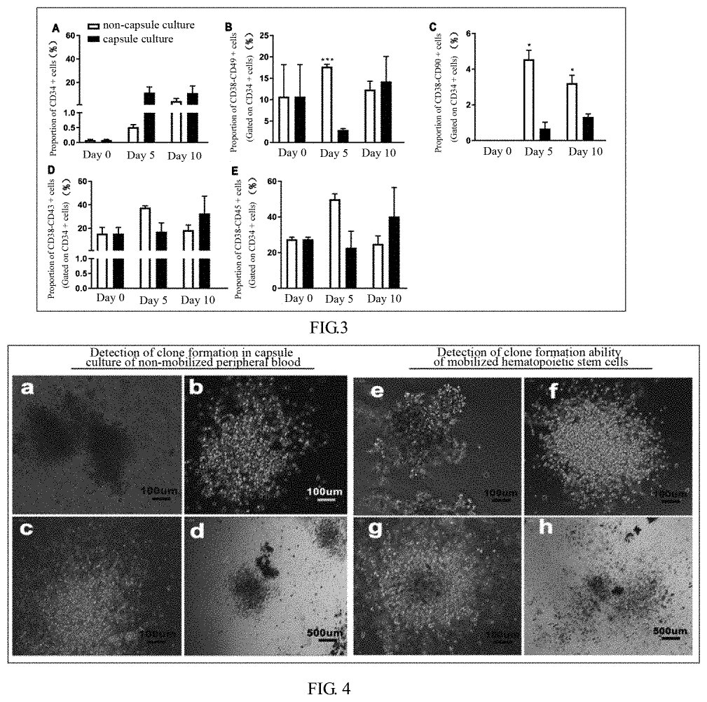

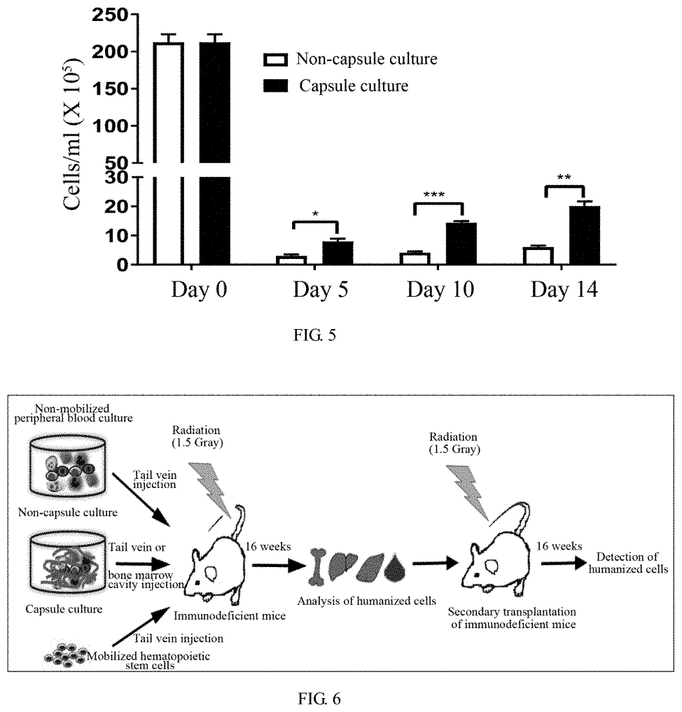

According to the morphologies of the clones, 200-300 CD34⁺ cells were sorted and cultured in a methylcellulose semi-solid medium containing a 20 ng/mL hematopoietic growth factor SCF, 20 ng/mL IL-3, 20 ng/mL IL-6, 20 ng/mL G-CSF, 20 ng/mL GM-CSF, 20 ng/mL TPO, and 3 U/mL EPO for about 2 weeks, and the formation of various hematopoietic CFU was detected. According to the morphological characteristics such as the structures formed by hematopoietic, cell size, color, and refractive index, the formation of various hematopoietic cell colonies was judged and counted. The results are shown in FIG. 6. FIG. 6 shows burst erythroid, megakaryocyte, granulocyte-macrophage, erythroid-granulocyte-macrophage/megakaryocyte hematopoietic colonies generated by culturing the paving stone-shaped clones obtained from non-mobilized peripheral blood in the hematopoietic growth factor-containing methylcellulose semi-solid medium for about 2 weeks. The detection of CFU formation ability of cells is shown in FIG. 4.

### 6. Growth Potential Analysis of the Cells in Capsule Culture System

Non-mobilized peripheral blood mononuclear cells were cultured equally in capsule and non-capsule culture system with a serum-free hematopoietic stem cell proliferation medium SFEM (STEMCELL TECHNOLOGY) containing 20-200 ng/mL SCF, 20-200 ng/mL FLT3L, 10-20 ng/mL IL-3, 10-20 ng/mL IL-6, 10-100 ng/mL TPO, 2-10 ng/mL VEGF, and 5-30 ug/ml vitamin C, the medium was replaced every 2 days, the culture was performed for about 14 days, the cells were counted, and the proliferation of the cells was calculated. The detection of growth of cells in different culture systems is shown in FIG. 5.

### 7. Detection of In Vivo Self-Renewal and Multi-Lineage Differentiation Potential of Cells Obtained in Capsule Culture System and Non-Capsule Culture System

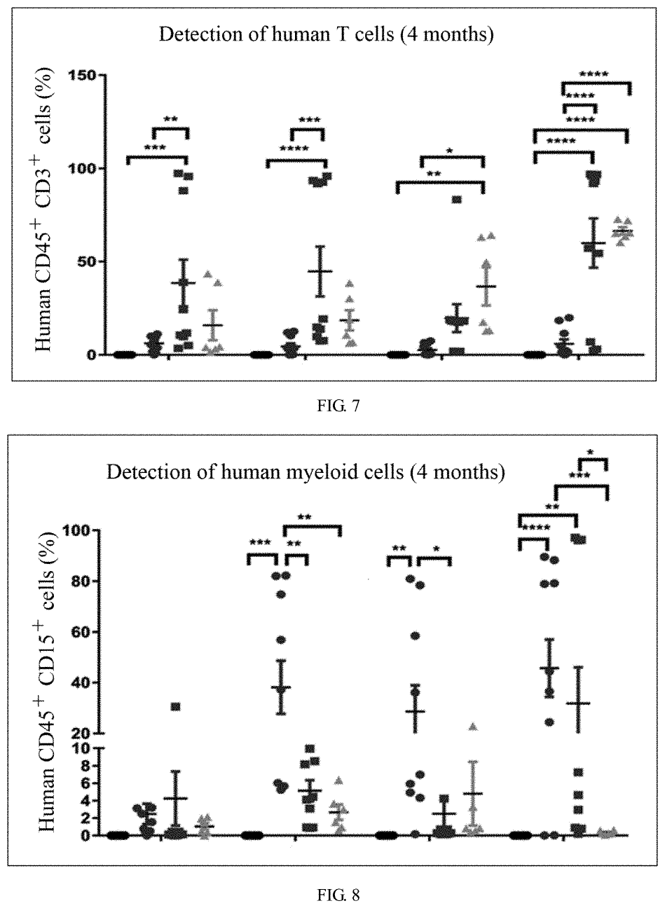

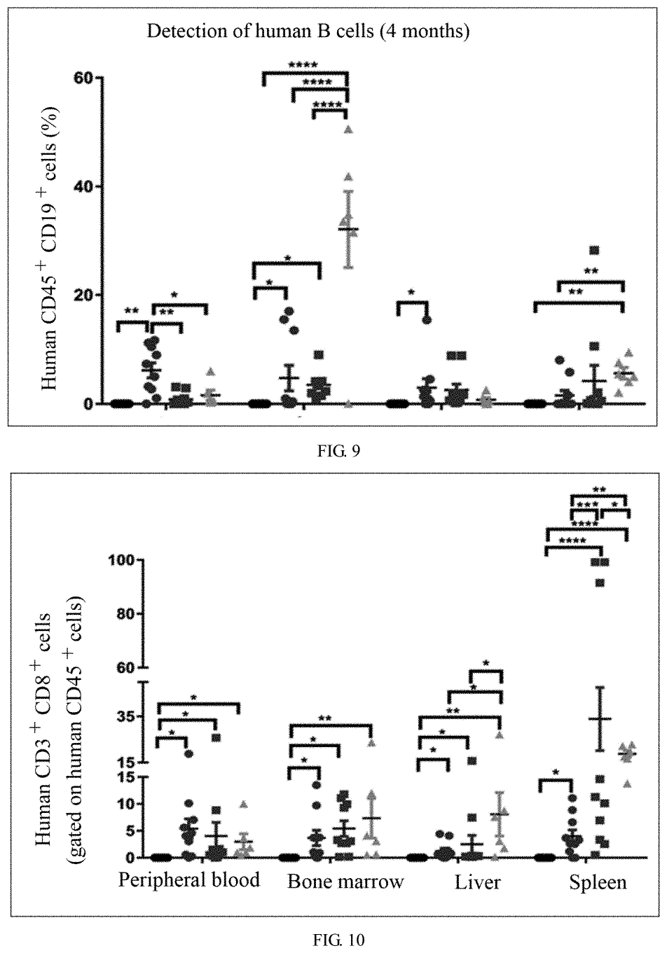

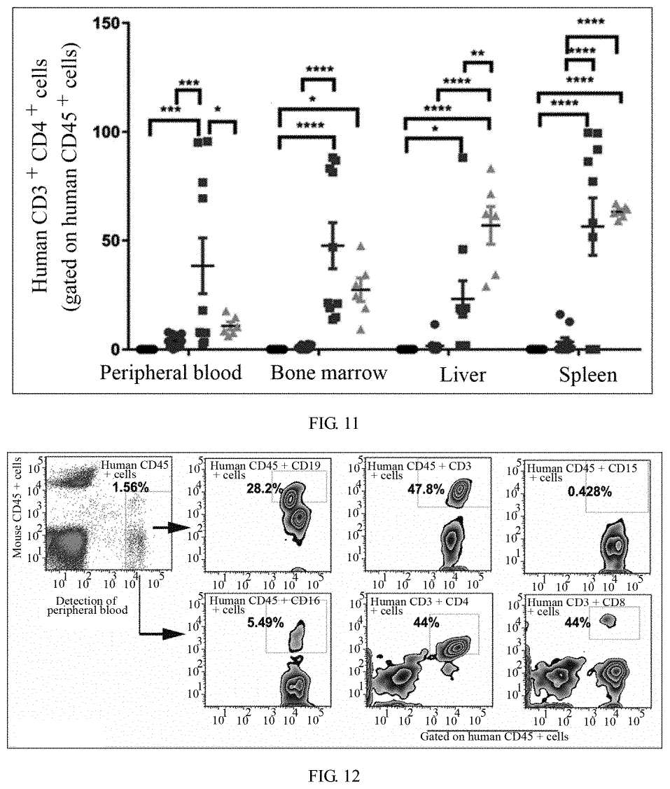

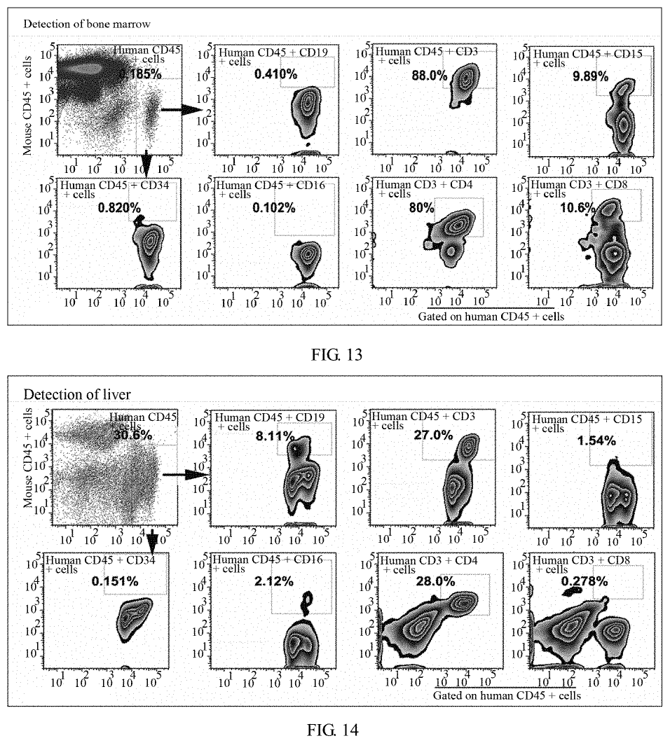

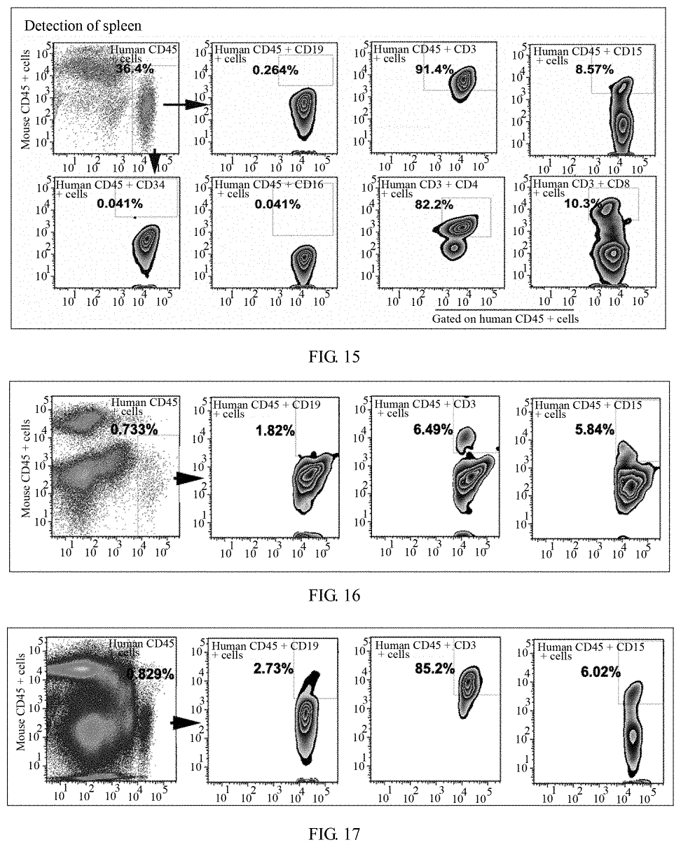

The mononuclear cells in non-mobilized peripheral blood were cultured in the capsule culture system and the non-capsule culture system for about 2 weeks, and then transplanted to immunodeficient mice. The self-renewal and multi-lineage differentiation potential of the cells were estimated. The chimerism of human cells in mice was periodically detected, including peripheral blood, bone marrow, spleen, liver and other organs, the cell types include human T cells, B cells, and myeloid cells, and the T cells include Th1 and Th2 types. The detection results of in vivo self-renewal and multi-lineage differentiation abilities of capsule cultured and non-capsule cultured non-mobilized peripheral blood cells are shown in FIGS. 6-15.

### 8. Detection of Long-Term In Vivo Self-Renewal and Multi-Lineage Differentiation Potential of Cells Obtained in Capsule Culture System and Non-Capsule Culture System

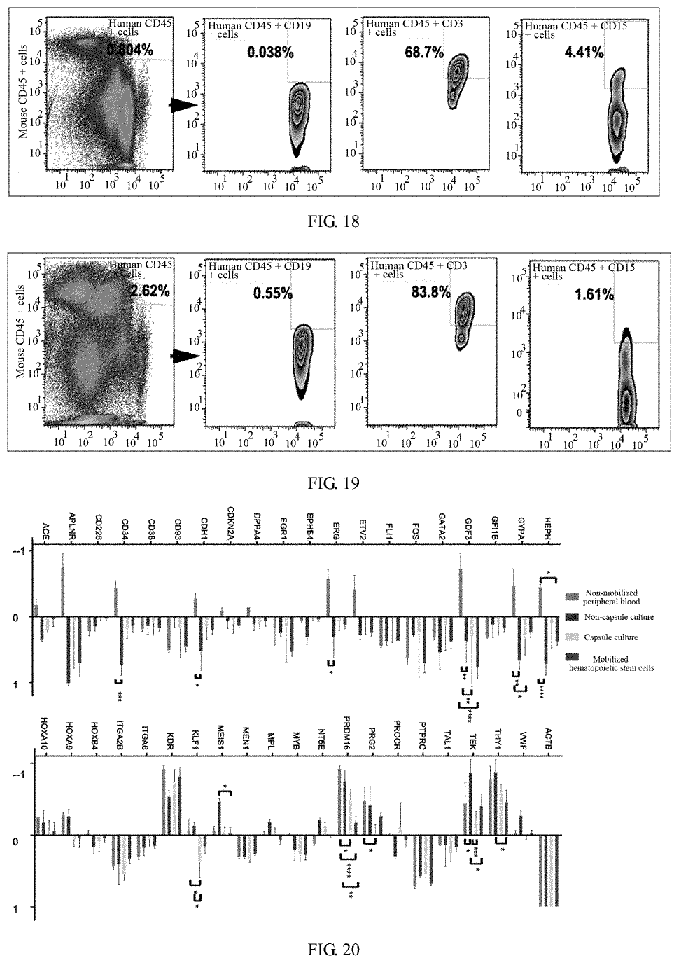

The presence of human cells in the transplanted mice indicates that the transplanted cells in capsule culture system have the self-renewal and multi-lineage differentiation potential. After four months after the first transplantation, bone marrow cells were taken out for second transplantation to detect the long-term self-renewal and multi-lineage differentiation potential of the cells in capsule culture system. After 1 month, 2 months, 3 months and 4 months, the human cell content was detected, specifically including peripheral blood, bone marrow, spleen, liver and other organs, and the cell types include human T cells, B cells, and myeloid cells. The results are shown in FIGS. 16-19.

### 9. Detection of Regulatory Mechanism of Cells in Capsule Culture System at Molecular Level with Transcriptome Sequencing

The specific steps were as follows: after the total RNA was extracted from a sample and DNA was digested with DNase I, eukaryotic mRNA was enriched with magnetic beads with Oligo (dT), a fragmentation reagent was added to break the mRNA into short fragments in Thermomixer, the fragmented mRNA was used as a template to synthesize one-strand cDNA, and then a two-strand synthesis reaction system was prepared to synthesize two-strand cDNA, the two-strand cDNA was purified and recovered by a kit, the sticky end was repaired, a base “A” was added to the 3′ terminal of cDNA and ligated with a linker, then the sizes of the fragments were selected, and finally PCR amplification was performed; after the constructed library passed a quality inspection by an Agilent 2100 Bioanalyzer and an ABI StepOnePlus Real-Time PCR System, the constructed library was then sequenced using an Illumina HiSeq™ 2000 sequencer.

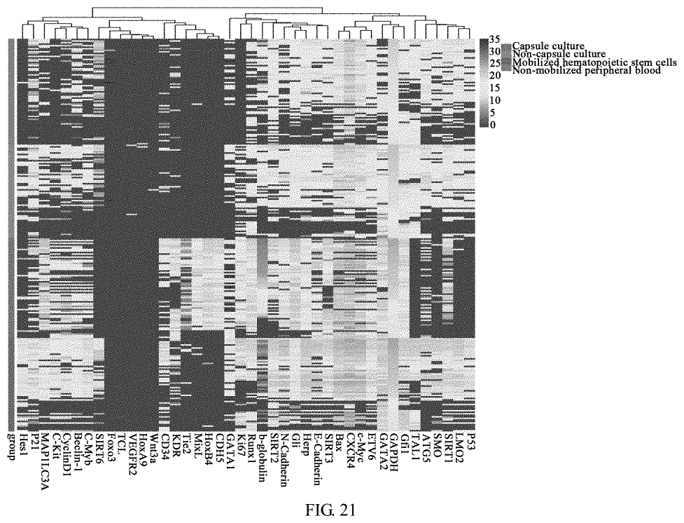

Information analysis of data obtained by Illumina HiSeq™ 2000 sequencing was performed, and raw reads of raw data were subjected to quality control (QC) to determine whether the sequencing data was suitable for subsequent analysis or not. The clean reads obtained by filtering were aligned to a reference sequence. After the alignment was completed, it was judged whether the alignment results passed a QC of alignment by counting the alignment rate and the distribution of reads on the reference sequence, etc. If the alignment results passed the QC of alignment, a series of subsequent analysis including quantitative analysis of genes and transcripts, gene expression level-based analysis of various items (principal components, correlation, condition-specific expression, differential gene screening, etc.), exon quantification, optimization of gene structure, alternative splicing, prediction and annotation of new transcripts, SNP detection, Indel analysis, gene fusion, etc. was performed, and the screened differentially expressed genes among the samples were subjected to key transcription factor mining analysis. The results are shown in FIG. 20.

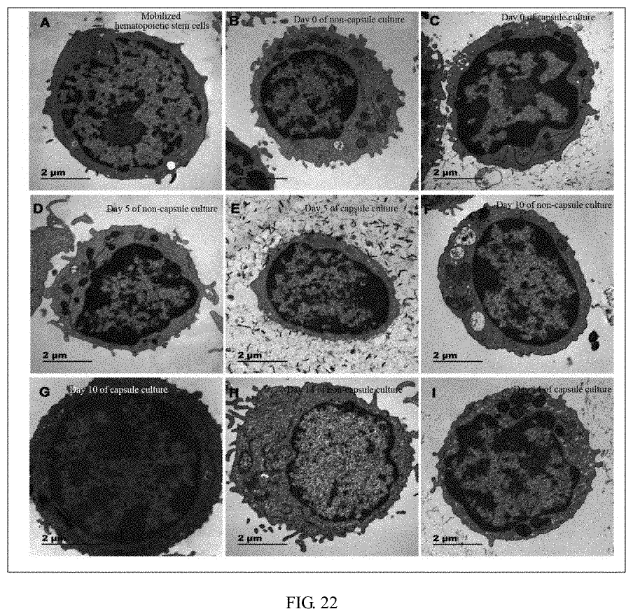

10. Detection of expression of key hematopoietic transcriptional regulatory factors in capsule cultured and non-capsule cultured non-mobilized peripheral blood cells by high-throughput fluorescent quantitative PCR. The primer sequence is shown in a typing sequence list. The results are shown in FIG. 21.

### 11. Detection of Morphologies and Internal Structural Characteristics of Cells by Scanning and Transmission Electron Microscopy

The ultrastructures of cells were analyzed by a transmission electron microscopy (TEM). The samples were fixed with a 2.5% glutaraldehyde solution for more than 4 h. After being washed with the calcium ion and magnesium ion-free phosphate buffer solution, the samples were treated with 1% osmic acid for 1 h, and then washed 2-3 times with distilled water. After being fixed in 2% uranium acetate, the cells were dehydrated in ethanol having a series of concentrations of 50%, 70%, 90%, and 100% for 10-15 min each time, and finally, the cells were soaked twice in 100% acetone for 10-15 min each time. After infiltration, retention, polymerization, and staining with a lead uranyl acetate citric acid solution, the internal structures of the cells were observed by the transmission electron microscopy TEM (Tecnai Spirit) at low temperature. The results are shown in FIG. 22.

What is claimed is:

1. A method for preparing heterogeneous hematopoietic stem and progenitor cells using non-mobilized peripheral blood, the method comprising the following steps:
(1) source and preparation of initiating cells
using normal peripheral blood without mobilizing drug treatment to obtain a blood product, removing erythrocytes from the obtained blood product by using a lymphocyte separation solution or an erythrocyte lysing solution, and washing the obtained mononuclear cells 2 to 3 times with a calcium ion and magnesium ion-free phosphate buffer solution to be ready for use as a source of cells to be initially cultured;
(2) preparation and culture of heterogeneous hematopoietic stem and progenitor cell clones
capsuling the above obtained mononuclear cells with hydrogel as a cell culture material and obtaining a capsule culture system, wherein, the cells are washed once with a 10% sucrose solution, re-suspended with 20% sucrose, capsuled with hydrogel, seeded in a well plate, and cultured in a culture medium, the culture medium being replaced every 2 to 3 days, so that clones with different morphologies appear;
(3) detection of heterogeneity of hematopoietic stem and progenitor cell clones by single cell sequencing
selecting single cell in the clones according to morphological characteristics, performing single cell sequencing, extracting single-cell RNAs, enriching eukaryotic mRNAs with magnetic beads with Oligo, synthesizing cDNAs using fragmented mRNAs as templates, purifying and recovering the obtained cDNAs by a kit, constructing a library by PCR amplification, sequencing the constructed library, detecting transcription expression of single-cell sequencing, analyzing gene expression, optimization of genetic structure, alternative splicing, prediction and annotation of new transcripts, and SNP detection according to a number of reads obtained by gene sequencing, and screening out genes that are differentially expressed among samples from gene expression results;
(4) surface molecule expression of heterogeneous hematopoietic stem and progenitor cell clones
growing various clones in the capsule culture system until each clone contains 30 to 80 cells, dispersing and mixing the system, performing digestion with an ethylenediamine tetraacetic acid digestive solution, passing the digested product through a 70 um mesh sieve, performing centrifugation to harvest cells, and detecting surface molecule expression of hematopoietic stem and progenitor cells in the harvested cells by using flow cytometry, including CD34, CD43, CD45, and CD90;
(5) detection of in vitro differentiation potential
selecting clones of several different morphologies appearing in the capsule culture system according to shapes of the clones, sorting 200 to 300 targeted cells for the clones, conducting a CFU experiment in a growth factor-containing methylcellulose semi-solid medium, and detecting multi-directional differentiation potential of different clones, including burst erythroid colonies, generally small erythroid colonies, granulocyte colonies, granulocyte-macrophage colonies, and erythroid-granulocyte-macrophage mixed cell colonies;
(6) detection of growth potential of the cells in capsule culture system
equally seeding non-mobilized peripheral blood mononuclear cells in capsule and non-capsule culture systems, conducting a growth potential study experiment in a medium containing a hematopoietic stem and progenitor cell growth factor, and detecting self-renewal potential of the different culture systems.
(7) detection of expression of transcription factors of hematopoietic stem cells in capsule culture system
studying biological characteristics of a whole cell population in the capsule cell culture system at a molecular level, detecting change of cells in the capsule culture system at a transcriptome level through RNA sequencing, especially hematopoietic stem cell-related transcription factors, signaling pathways, and microenvironment-related regulating factors; and
(8) detection of in vivo hematopoietic differentiation potential of whole cell population in whole capsule cell culture system
subjecting a cell population formed by dispersing various hematopoietic colonies to a transplantation experiment, detecting long-term in vivo self-renewal and multi-directional differentiation potential of the cells, and periodically detecting implantation of humanized cells in mice, where cells that are non-capsule cultured under the same conditions are used as a control.

2. The method for preparing the heterogeneous hematopoietic stem and progenitor cells using the non-mobilized peripheral blood according to claim 1, wherein in step (2), the culture medium consists of 20-150 ng/ml stem cell growth factor SCF, 20-150 ng/ml FMS-like tyrosine kinase 3 ligand antibody, 20-100 ng/ml thrombopoietin TPO, 10-50 ng/ml interleukin 6 IL6, 10-50 ng/ml interleukin 3 IL3, 2-10 ng/ml vascular growth factor VEGF, 10-20 ug/ml vitamin C, and puromycin derivative StemRegenin1.

3. The method for preparing the heterogeneous hematopoietic stem and progenitor cells using the non-mobilized peripheral blood according to claim 1, wherein in step (2), the clones of different morphologies appearing upon culturing comprises dense clones, vascular clones, paving stone-shaped clones, and freely dispersed clones.

4. The method for preparing the heterogeneous hematopoietic stem and progenitor cells using the non-mobilized peripheral blood according to claim 1, wherein in step (3), GO function significance enrichment analysis and pathway significance enrichment analysis are performed based on the genes that are differentially expressed to analyze cell clusters of principal components of single cells, so as to detect the heterogeneity of said various clones.

5. The method for preparing the heterogeneous hematopoietic stem and progenitor cells using the non-mobilized peripheral blood according to claim 1, wherein in step (7), the transcription factors comprise CD34, RUNX1, GATA2, c-MYC, HOXA9, HOXB4, GATA1, and TIE2; the signal pathways mainly comprise genes regulating self-renewal, multi-lineages potential and metabolism state; and the microenvironment-related regulating factors are mainly homing and cell adhesion-related genes.
